(19) United States
(12) Patent Application Publication
(10) Pub. No.: US 2026/0265667 A1
(43) Pub. Date: Sep. 10, 2026

(54) TEMPERATURE OSCILLATION DEVICE FOR PHYSIOLOGICALLY RELEVANT CELL CULTURE SYSTEMS

(71) Applicant: University of Kentucky Research Foundation, Lexington, KY (US)

(72) Inventors: Elizabeth Schroder Stumpf, Lexington, KY (US); Brian P. Delisle, Lexington, KY (US)

(21) Appl. No.: 19/559,494

(22) Filed: Mar. 6, 2026

Related U.S. Application Data
(60) Provisional application No. 63/767,906, filed on Mar. 6, 2025.

(51) Int. Cl. C12M 1/00; C12Q 1/6897

(52) U.S. Cl. CPC C12M 41/14; C12Q 1/6897

(57) ABSTRACT

A temperature oscillation overlay device configured for removable integration with an existing tissue culture incubator having a native temperature controller and a native heating element includes a temperature sensing system physically independent of the native temperature controller, the temperature sensing system comprising at least one temperature sensor configured for placement within an interior chamber of the incubator, a central microcontroller configured to receive temperature measurements obtained by the at least one temperature sensor, an independent internal heating element, a power modulation interface configured to selectively interrupt or modulate electrical power delivered to the internal heating element; and a programmable waveform engine executed by the central microcontroller to generate a biologically defined time-varying temperature profile. Associated methods of entraining circadian gene expression in cultured cells and detecting circadian regulatory elements in a gene promoter utilize the temperature oscillation overlay device.

### CROSS-REFERENCE TO RELATED APPLICATIONS

This application claims priority to U.S. Provisional Application 63/767,906, filed Mar. 6, 2025, which is incorporated by reference herein in its entirety.

### TECHNICAL FIELD

Embodiments of the present disclosure generally relate to temperature oscillation overlay devices configured for removable integration with an existing tissue culture incubator as well as methods of entraining circadian gene expression in cultured cells and/or detecting circadian regulatory elements in a gene promoter utilizing the same.

### BACKGROUND

Temperature oscillations are critical in regulating physiological processes across various organisms. In diurnal mammals, including humans, core body temperature fluctuates throughout the day, typically reaching its lowest point in the early morning and peaking in the late afternoon or evening. These temperature rhythms are tightly interconnected with the circadian clock system, influencing gene expression, metabolic processes, and cellular functions. Moreover, disruptions in temperature rhythms have been linked to various health issues, including sleep disorders, metabolic dysfunctions, and even cancer progression. Understanding and accurately replicating these temperature oscillations in experimental settings is critical for advancing our knowledge of chronobiology and developing more effective therapies for circadian rhythm-related disorders. However, current solutions for synchronizing circadian rhythms in cell culture provide only a transient synchronizing stimulus, resulting in rapid dampening of circadian oscillations within 3-5 days, high inter-replicate variability in period length and acrophase timing, low-amplitude circadian gene expression, and low baseline gene expression. Specifically, synchronizing circadian rhythms in cell culture is typically currently achieved using primarily serum shock which is a technique where cells are temporarily exposing to a high concentration of serum, for example 50% horse serum or fetal bovine serum (FBS), for a short duration on the order of 2 hours. This “shock” synchronizes the expression of circadian genes between cells, allowing researchers to study clock mechanisms in vitro, but as noted there is typically rapid dampening of circadian oscillations within 3-5 days along with other noted deficiencies. Additional synchronization methods include dexamethasone treatment, forskolin treatment, and temperature pulse methods, all of which share the common limitation of providing only a transient synchronizing stimulus rather than a continuous sustained zeitgeber, resulting in the rapid dampening of circadian oscillations and experimental limitations noted above. No commercially available device currently enables researchers to introduce programmable, physiologically relevant, continuous temperature oscillations into existing incubator infrastructure in a non-destructive add-on format, representing a significant unmet need in chronobiology, cell biology, and biomedical research broadly.

### SUMMARY

As such, there are needs for solutions to maintain circadian oscillations in cells of a cell culture for an extended time. As temperature cycles can entrain circadian rhythms in peripheral tissues, acting as a powerful zeitgeber (time-giver) alongside light-dark cycles, precise temperature control may be leverage to maintain circadian oscillations. The present disclosure provides devices which can provide precise, programmable temperature oscillations and be easily adapted for pre-existing tissue culture incubators. This innovation will significantly enhance the capabilities of standard incubators, allowing researchers to more accurately mimic physiological temperature fluctuations and study their effects on cellular processes. The devices of the present disclosure also allow for further temperature profiling as well as cellular study to be readily achievable. Specifically, the temperature oscillation overlay device of the present disclosure addresses the unmet need for a non-destructive, add-on solution that integrates with existing tissue culture incubator infrastructure without permanent modification, enabling researchers to introduce physiologically relevant temperature dynamics into their existing experimental systems without capital investment in new equipment. Beyond circadian research, the programmable nature of the temperature oscillation overlay device enables application to any temperature-sensitive biological process, including drug development and chronopharmacology, organoid and tissue engineering applications, fever and hypothermic modeling, and cardiac and metabolic rhythm research, making it a broadly applicable platform technology for biomedical research and translational applications.

Embodiments of the present disclosure relate to a temperature oscillation overlay device configured for removable integration with an existing tissue culture incubator having a native temperature controller and native heating element. The device includes temperature sensing system physically independent of the native temperature controller with the temperature sensing system including at least one temperature sensor configured for placement within an interior chamber of the incubator and a temperature sensing system microcontroller configured to receive data from the temperature sensor. The device also includes a central microcontroller configured to receive temperature measurements obtained by the at least one temperature sensor from the temperature sensing system microcontroller. Further, the device includes an independent internal heating element configured for placement within the interior chamber of the incubator and operated entirely separately from and without modification of the native heating element or native temperature controller. The device also includes a power modulation interface comprising a power modulation interface microcontroller configured to selectively modulate electrical power delivered exclusively to the independent internal heating element without modification of the incubator's internal control circuitry. The device further includes a programmable waveform engine executed by the central microcontroller and configured to generate a biologically defined time-varying temperature profile. Finally, the central microcontroller controls power delivery to the independent internal heating element via the power modulation interface in a closed feedback loop independent of the native temperature controller to superimpose the oscillatory temperature profile wherein temperature increases are achieved by increasing power delivery to the independent internal heating element and temperature decreases are achieved by reducing or ceasing power delivery to the independent internal heating element, with the rate of passive thermal equilibration toward the stable thermal baseline controlled by the central microcontroller to conform to the descending phases of the biologically defined time-varying temperature profile.

Embodiments of the present disclosure also relate to a method of entraining circadian gene expression in cultured cells. The method includes maintaining the cells in a standard tissue culture incubator and then superimposing using the temperature oscillation overlay device of the present disclosure a continuous oscillatory temperature profile having a period of 18 to 30 hours and an amplitude between 0.5° C. and 3° C. Further, the method includes maintaining the oscillatory temperature profile for at least five consecutive circadian cycles. The oscillatory temperature profile functions as a sustained zeitgeber that increases circadian amplitude and reduces inter-replicate variability relative to serum shock synchronization.

Embodiments of the present disclosure also relate to a method of detecting circadian regulatory elements in a gene promoter. The method includes introducing into cultured cells a reporter construct comprising a promoter region operatively linked to a reporter gene and then maintaining the cultured cells in a standard tissue culture incubator. The method further includes superimposing using the temperature oscillation overlay device of the present disclosure a continuous oscillatory temperature profile having a period of 18 to 30 hours and an amplitude between 0.5° C. and 3° C. Further, the method includes maintaining the oscillatory temperature profile for at least five consecutive circadian cycles and measuring reporter gene expression over time. Differences in amplitude, period, acrophase, or baseline expression of reporter gene activity under the superimposed continuous oscillatory temperature profile identify the presence or functional contribution of circadian regulatory elements under study.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 is a schematic illustration of a temperature oscillation overlay device in accordance with one or more embodiments of the present disclosure;

FIG. 2 is a schematic illustration of a temperature oscillation overlay device including an auxiliary temperature control unit in accordance with one or more embodiments of the present disclosure;

FIG. 3 is a graph of an oscillating temperature profile in accordance with one or more embodiments of the present disclosure;

FIG. 4A is a graph of Lumicycle data from C2C12 myotubes expressing promoter-luciferase constructs for the clock genes Bmal1 and Per1 with serum shock to align clocks of cells;

FIG. 4B is a graph of Lumicycle data from C2C12 myotubes expressing promoter-luciferase constructs for the clock genes Bmal1 and Per1 with temperature oscillation to align clocks of cells in accordance with one or more embodiments of the present disclosure;

FIG. 5A is a graph of Lumicycle data from C2C12 myotubes expressing promoter-luciferase constructs for GJA1 with temperature oscillation to align clocks of cells in accordance with one or more embodiments of the present disclosure;

FIG. 5B is a graph of Lumicycle data from C2C12 myotubes expressing promoter-luciferase constructs for GJA1 with temperature oscillation to align clocks of cells in accordance with one or more embodiments of the present disclosure; and

FIG. 5C is a graph of Lumicycle data from C2C12 myotubes expressing promoter-luciferase constructs for GJA1 with temperature oscillation to align clocks of cells in accordance with one or more embodiments of the present disclosure.

### DETAILED DESCRIPTION

Embodiments of the present disclosure generally relate to a temperature oscillation overlay device configured for removable integration with an existing tissue culture incubator. The temperature oscillation overlay device generates precise, programmable temperature oscillations within standard academic or commercial research incubators to simulate physiological circadian and other biological temperature rhythms in vitro. This flexibility allows existing academic or commercial research incubators to be leveraged to generate temperature conditions conducive to activation of circadian or other biological temperature rhythm induced responses in cell cultures. Specifically, standard tissue culture incubators maintain cells at a fixed temperature, typically 37° C. However, warm-blooded organisms experience daily circadian temperature fluctuations typically of approximately 1-2° C. driven by biological clocks. This discrepancy between static in vitro conditions and dynamic in vivo temperature environments is a significant source of experimental irreproducibility in chronobiology, cell biology, and biomedical research. The temperature oscillation overlay device in accordance with the present disclosure allows for dynamic in vitro temperature environments to more closely mimic in vivo conditions, thereby reducing or eliminating the significant source of experimental irreproducibility in chronobiology, cell biology, and biomedical research. Beyond basic research applications, the ability to introduce physiologically relevant temperature dynamics into standard cell culture systems has broad implications for drug development, where circadian timing is increasingly recognized as a critical variable in drug efficacy and toxicity, and for organoid and tissue engineering applications, where dynamic temperature environments more faithfully replicate the in vivo conditions under which complex tissue architecture and function develop. By introducing the dimension of time into cell culture through programmable temperature oscillation, the present device enables a more physiologically accurate experimental platform across a wide range of biomedical research and translational applications.

The temperature oscillation overlay device is configured to generate precise, programmable temperature oscillations within standard academic or commercial research tissue culture incubators to simulate physiologically relevant biological temperature rhythms in vitro, without permanent modification of the existing tissue culture incubator. The temperature oscillation overlay device operates by establishing a stable thermal baseline via the native temperature controller of the existing tissue culture incubator and superimposing a programmable oscillatory temperature profile onto that baseline via an independent internal heating element positioned within the interior chamber of the existing tissue culture incubator, entirely separately from and without modification of the native heating element or native temperature controller. Applications of the temperature oscillation overlay device include but are not limited to circadian rhythm research, ultradian and infradian rhythm research, fever and hypothermic modeling, heat shock and stress response studies, drug development and chronopharmacology, organoid and tissue engineering applications, and cardiac and metabolic rhythm research.

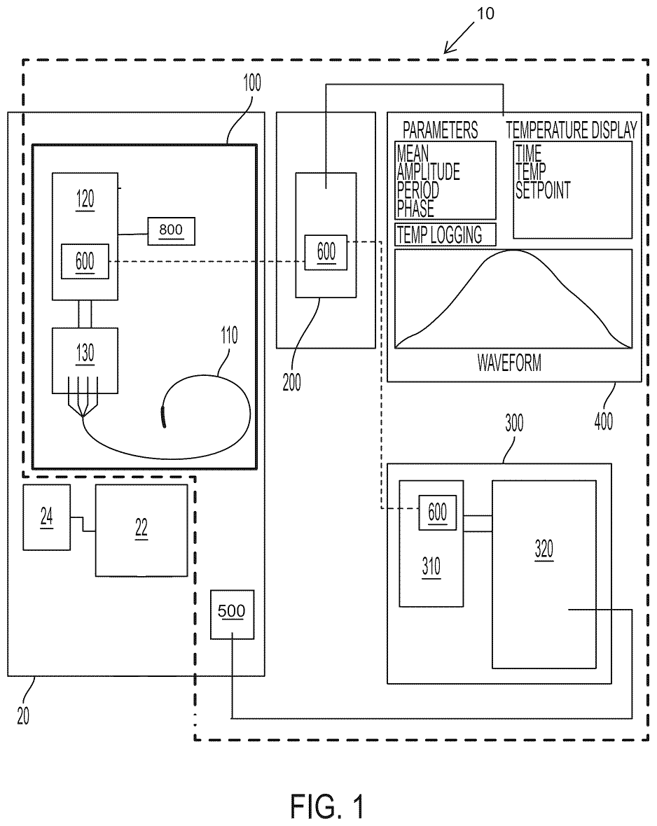

In accordance with embodiments of a temperature oscillation overlay device 10 configured for removable integration with an existing tissue culture incubator 20 having a native temperature controller 24 and native heating element 22 and with reference to FIG. 1, the device 10 comprises a temperature sensing system 100, a central microcontroller 200, an independent internal heating element 500, a power modulation interface 300, and a programmable waveform engine 400. The temperature sensing system 100 is physically independent of the native temperature controller 24 and the temperature sensing system 100 comprises at least one temperature sensor 110 configured for placement within an interior chamber of the incubator 20 and a temperature sensing system microcontroller 120 configured to receive data from the temperature sensor 110. The central microcontroller 200 is configured to receive temperature measurements obtained by the at least one temperature sensor 110 from the temperature sensing system microcontroller 120. Further, the independent internal heating element 500 is configured for placement within the interior chamber of the incubator 20 and is operated entirely separately from and without modification of the native heating element 22 or internal control circuitry of the existing tissue culture incubator 20. The power modulation interface 300 comprises a power modulation interface microcontroller310 and a power relay 320, wherein the power modulation interface microcontroller 310 is configured to selectively modulate electrical power delivered exclusively to the independent internal heating element 500 without modification of the internal control circuitry of the existing tissue culture incubator 20. The programmable waveform engine 400 is executed by the central microcontroller 200 and is configured to generate a biologically defined time-varying temperature profile. Finally, the central microcontroller 200 controls power delivery to the independent internal heating element 500 via the power modulation interface 300 in a closed feedback loop independent of the native temperature controller 24 of the existing tissue culture incubator 20 to superimpose the oscillatory temperature profile onto a stable thermal baseline established by setting the native temperature controller 24 to the minimum temperature of the desired oscillation profile.

Having generally described the various components of the temperature oscillation overlay device 10, each component and sub-system will be described in further detail. As previously indicated and with reference to FIG. 1, the temperature oscillation overlay device 10 is configured for removable integration with an existing tissue culture incubator 20 by modulating power delivery to the independent internal heating element 500, which operates entirely separately from and without modification of the native heating element 22 or native temperature controller 24. The existing tissue culture incubator 20 is configured with its native temperature controller 24 set to maintain the minimum temperature of the desired oscillation profile, establishing a stable thermal baseline upon which the independent internal heating element 500 superimposes the programmable oscillatory temperature profile. The methods and techniques to affect this modulation of the independent internal heating element 500 independent of the native temperature controller 24 are discussed in further detail in conjunction with disclosure of the power modulation interface 300.

The temperature oscillation overlay device 10 may be implemented in a variety of existing tissue culture incubators 20. As the device 10 requires no permanent modification to the existing tissue culture incubator 20 and operates via the independent internal heating element 500 placed within the interior chamber of the incubator 20 without interference with the native heating element 22 or native temperature controller 24, implementation is considered agnostic to the specific type of incubator. For example, the temperature oscillation overlay device 10 may be implemented in both water-jacketed and air-jacketed type incubators.

The temperature sensing system 100 includes at least one temperature sensor 110 and a temperature sensing system microcontroller 120. In accordance with one or more embodiments, the entire temperature sensing system 100 is positioned within an interior chamber of the existing tissue culture incubator 20. In further embodiments, at least the at least one temperature sensor 110 is positioned within an interior chamber of the existing tissue culture incubator 20. Positioning the temperature sensing system 100 within the interior chamber of the existing tissue culture incubator 20 allows for acquisition and monitoring of the temperature of the environment within the existing tissue culture incubator 20. It will be appreciated that the at least one temperature sensor 110 should be positioned within the existing tissue culture incubator 20 such that an accurate reading of the temperature experienced by cells culturing within the existing tissue culture incubator 20 is recorded. Additionally, it is noted that at least one temperature sensor 110 is placed within the existing tissue culture incubator 20 in a manner that does not interfere with cell culture operations.

In various embodiments, at least one temperature sensor 110 may comprise a thermistor, resistance temperature detector, or diode-based sensor. A thermistor is a highly sensitive, semiconductor-based resistor whose electrical resistance changes significantly and predictably in response to temperature variations. An RTD (Resistance Temperature Detector) is a passive, highly accurate, and stable sensor that measures temperature by correlating it with a predictable increase in electrical resistance in a metal element as it gets hotter. In accordance with one or more specific embodiments, the at least one temperature sensor 110 comprises a platinum resistance temperature detector. A diode-based temperature sensor measures temperature by monitoring the linear decrease in a silicon diode's forward voltage drop (V_{f}) as temperature rises such that driven by a constant current, the forward voltage (V_{f}) decreases as temperature increases.

In one or more embodiment, the temperature sensing system 100 further comprises a signal-conditioning amplifier 130. Specifically, the temperature sensing system 100 in accordance with one or more embodiments includes the at least one temperature sensor 110 and a signal-conditioning amplifier 130 configured to process the signal from the at least one temperature sensor 110. The at least one temperature sensor 110 is arranged to generate an analog voltage or current signal indicative of a measured temperature. The output of the at least one temperature sensor 110 is electrically connected to an input of the signal-conditioning amplifier 130. In one or more embodiments, the signal-conditioning amplifier 130 is a high-impedance, low-offset operational amplifier (op-amp) configured as a voltage follower, buffer, or differential gain stage. The signal-conditioning amplifier 130 acts to amplify the low-level signal from the at least one temperature sensor 110, improve signal-to-noise ratio, and provide a buffered, low-impedance output signal proportional to the temperature for subsequent processing stages such as an analog-to-digital converter or the temperature sensing system microcontroller 120.

In accordance with one or more embodiments, to enhance accuracy, the signal-conditioning amplifier 130 and associated circuit may include feedback components such as resistors to set a specific gain and, in certain embodiments, a feedback network with temperature-dependent resistive elements to provide active temperature compensation, reducing nonlinearities and thermal drift of the signal-conditioning path.

In accordance with various embodiments, the at least one temperature sensor 110 has an accuracy of ±0.3, ±0.2, ±0.1, ±0.05, ±0.03, or ±0.01 across an operating range of at least −70 to 260° C., at least −70 to 200° C., −70 to 150° C., −70 to 100° C., −70 to 75° C., −70 to 50° C., −70 to 42° C., −20 to 260° C., at least −20 to 200° C., −20 to 150° C., −20 to 100° C., −20 to 75° C., −20 to 50° C., −20 to 42° C., 0 to 260° C., at least 0 to 200° C., 0 to 150° C., 0 to 100° C., 0 to 75° C., 0 to 50° C., 0 to 42° C., 15 to 260° C., at least 15 to 200° C., 15 to 150° C., 15 to 100° C., 15 to 75° C., 15 to 50° C., 15 to 42° C., 30 to 260° C., at least 30 to 200° C., 30 to 150° C., 30 to 100° C., 30 to 75° C., 30 to 50° C., or 30 to 42° C.

In accordance with various embodiments, the at least one temperature sensor 110 measures temperature at a frequency of once every 0.1 seconds, 0.5 seconds, 1 second, 2 seconds, 3 seconds, 4 seconds, 5 seconds, 6 seconds, 7 seconds, 8 seconds, 9 seconds, 10 seconds, 15 seconds, 20 second, 25 seconds, 30 seconds, 35 seconds, 40 seconds, 45 seconds, 50 seconds, 55 seconds, 60 seconds, 2 minutes, 3 minutes, 5 minutes, 10 minutes, 12 minutes, 15 minutes, 18 minutes, 20 minutes, 22 minutes, 25 minutes, 28 minutes, or 30 minutes as well as all nested ranges subsumed by the expressly disclosed values and their intermediaries.

The temperature sensing system microcontroller 120 may be any suitable processing device capable of executing instructions to perform the operations described herein, such as system interchangeable referred to as a microprocessor, a microcontroller, a System-on-a-Chip (SoC), a digital signal processor (DSP), a field-programmable gate array (FPGA), or an application-specific integrated circuit (ASIC). In one embodiment, the temperature sensing system microcontroller 120 includes a central processing unit (CPU), a memory (e.g., RAM, ROM, Flash memory), and input/output (I/O) peripherals. The memory may store machine-readable instructions that, when executed by the CPU, cause the temperature sensing system microcontroller 120 to perform the methods and algorithms described in connection with the temperature sensing system microcontroller 120. One skilled in the art has familiarity with microcontrollers in general and is capable of selection of an appropriate microcontroller for governing the specific operational guidelines of the temperature sensing system microcontroller 120. While a specific microcontroller is described, it should be understood that the techniques described herein are not limited to a particular hardware configuration, and the components may be distributed across multiple devices or integrated into a single component.

In accordance with one or more embodiments, the temperature sensing system 100 may enter a sleep mode between temperature readings to enhance power efficient operation. Additionally, the frequency of temperature readings may be adjusted to reduce power consumption with a reduction in frequency of temperature readings resulting in lower total power consumption.

In accordance with one or more embodiments, the temperature sensing system 100 may be powered by one or more batteries. Powering the temperature sensing system 100 with a battery allows for positioning the temperature sensing system 100 entirely within the interior chamber of the existing tissue culture incubator 20. Accordingly, in one or more embodiments the at least once temperature sensor 110 is provided with battery power. Similarly, in one or more embodiments the temperature sensing system microcontroller 120 is provided with battery power for operation. In one or more embodiments, the battery is a lithium-ion battery. In one or more specific embodiments, the battery is rechargeable. In accordance with various embodiments, the battery may be selected to have sufficient energy capacity to power the temperature sensing system 100 for at least 1 day, at least 2 days, at least 3 days, at least 4 days, at least 5 days, at least 6 days, at least 7 days, at least 8 days, at least 9 days, at least 10 days, at least 11 days, at least 12, days at least 13, days, at least 14 days, at least 21 days, or at least 28 days without recharging or replacement.

The central microcontroller 200 serves as a centralized control hub to connect and coordinate the various sub-systems of the temperature oscillation overlay device 10. Specifically, the central microcontroller 200 receives temperature measurements obtained by the at least one temperature sensor 110 from the temperature sensing system microcontroller 120 as well as defined time-varying temperature profiles from the programmable waveform engine 400. The central microcontroller 200 compares the measured temperature readings from the temperature sensing system 100 and the target temperature from the programmable waveform engine 400 and determines appropriate instructions for the power modulation interface 300 to modulate power delivery to the independent internal heating element 500 to achieve the desired temperature adjustment within the interior chamber of the existing tissue culture incubator 20, independent of and without modification to the native temperature controller 24 or native heating element 22. In accordance with one or more embodiments, this comparison and adjustment is performed continuously in a closed feedback loop, wherein the central microcontroller 200 dynamically modulates power delivery to the independent internal heating element 500 to minimize the difference between the measured temperature and the target temperature defined by the programmable waveform engine 400, including during descending phases of the profile where power delivery is reduced or ceased and the rate of passive thermal equilibration is controlled to conform to the programmed waveform.

Similar to the temperature sensing system microcontroller 120, the central microcontroller 200 may be any suitable processing device capable of executing instructions to perform the operations described herein, such as a deviceinterchangeably referred to as a microprocessor, a microcontroller, a System-on-a-Chip (SoC), a digital signal processor (DSP), a field-programmable gate array (FPGA), or an application-specific integrated circuit (ASIC). In one embodiment, central microcontroller 200 includes a central processing unit (CPU), a memory (e.g., RAM, ROM, Flash memory), and input/output (I/O) peripherals. The memory may store machine-readable instructions that, when executed by the CPU, cause the central microcontroller 200 to perform the methods and algorithms described in connection with the central microcontroller 200. In accordance with one or more specific embodiments, the central microcontroller 200 comprises an ESP32 or equivalent microcontroller configured to receive wireless temperature measurements from the temperature sensing system microcontroller 120 via ESP-NOW protocol, execute the programmable waveform engine 400, and transmit control signals to the power modulation interface 300 to modulate power delivery to the independent internal heating element 500. One skilled in the art has familiarity with microcontrollers in general and is capable of selection of an appropriate microcontroller for governing the specific operational guidelines of the central microcontroller 200. While a specific microcontroller is described, it should be understood that the techniques described herein are not limited to a particular hardware configuration, and the components may be distributed across multiple devices or integrated into a single component.

The programmable waveform engine 400 is executed by the central microcontroller 200 and is configured to generate a biologically defined time-varying temperature profile. The biologically defined time-varying temperature profile represents a temperature profile which changes with respect to time and replicates or simulations a natural temperature profile expected to be experienced by cells in vivo. In accordance with various embodiments, the biologically defined time-varying temperature profile is representative of a circadian rhythm, an ultradian rhythm profile, an infradian rhythm profile, a fever profile, or a hypothermic profile.

A circadian rhythm temperature profile describes an endogenous, cycle of increasing and decreasing core body temperature (CBT). The circadian cycle in humans is roughly 24 hours. It represents a sinusoidal-like wave characterized by a minimum value termed bathy phase during sleep and a maximum value termed acrophase in the late afternoon/early evening. In humans the sinusoidal-like wave of temperature typically comprises an amplitude of roughly 0.2° C. to 1.0° C. In accordance with one or more embodiments, the circadian rhythm temperature profile generated by the programmable waveform engine 400 comprises an amplitude of 0.5° C. to 3° C. and a period of approximately 24 hours, including periods ranging from 18 to 30 hours, to simulate physiological circadian temperature dynamics in cultured cells. An ultradian rhythm temperature profile describes a biologically defined temperature cycle with a period shorter than 24 hours, including but not limited to cycles of approximately 90 minutes to 12 hours, relevant to cell cycle dynamics, metabolic oscillations, and other sub-circadian biological processes.

An infradian rhythm temperature profile describes a biologically defined temperature cycle with a period longer than 24 hours, relevant to modeling seasonal, weekly, or other extended biological rhythms.

A fever profile replicates a temperature cycle experienced in vivo during a fever. Specifically, the fever profile includes an elevated temperature where fevers commonly feature a rapid rise in temperature, a sustained plateau with fluctuating high temperatures, and a “breaking” phase as the fever subsides and temperature cools. In accordance with one or more embodiments, the fever profile comprises a rapid temperature rise to between 38° C. and 41° C., a sustained plateau phase, and a controlled return to baseline temperature.

A hypothermic profile reduces the temperature in the tissue culture incubator 20 to induce hypothermic conditions for the cells being cultured. In humans, a hypothermic temperature condition is typically defined by a core body temperature falling below 35° C. The hypothermic temperature profile may include reduction in temperature as well as controlled elevation of temperature to replicate conditions experienced by cells in vivo exposed to hypothermic conditions and naturally body responses to return to baseline core body temperature.

In accordance with one or more embodiments, the programmable waveform engine 400 is further configured to generate fully custom temperature profiles defined by user-specified parameters including waveform shape, amplitude, period, phase, and mean temperature, enabling application of the temperature oscillation overlay device 10 to any temperature-sensitive biological process beyond the preloaded physiological profile templates.

In accordance with one or more embodiments, the programmable waveform engine 400 permits user-defined adjustment of amplitude, period, phase, and mean temperature of the biologically defined time-varying temperature profiles. In accordance with one or more embodiments, the programmable waveform engine 400 further permits user-defined selection of waveform shape, including sinusoidal, square wave, ramp, and fully custom-defined profiles. Adjustment of one or more of the amplitude, period, phase, and mean temperature of the biologically defined time-varying temperature profiles allows for the biologically defined time-varying temperature profiles to be modified to experimentally replicate a vast array of in vivo cell conditions within the tissue culture incubator 20. For example, the amplitude may be adjusted to allow for circadian rhythm temperature profiles with both minimal or maximal temperature swings, fever profiles with differing peak temperatures, and hypothermic profiles with differing minimum temperatures. In accordance with one or more embodiments, the amplitude is adjustable within a range of 0.1° C. to 5° C., the period is adjustable from 17 hours to 30 hours, and the mean temperature is adjustable within the operating range of 30° C. to 50° C., thereby enabling the programmable waveform engine 400 to replicate a broad spectrum of physiologically relevant temperature dynamics across a wide range of biological research applications. Adjustment of period, phase, and/or mean temperature of the biologically defined time-varying temperature profiles provides similar versatility in the temperature profiles generated by the programmable waveform engine 400. In accordance with one or more embodiments, the programmable waveform engine 400 is further configured to coordinate temperature profiles across multiple tissue culture incubators 20 simultaneously, enabling experimental designs requiring phase-shifted or otherwise coordinated temperature oscillations across multiple independent cell culture systems.

In accordance with various embodiments, the biologically defined time-varying temperature profile may comprise a circadian rhythm with a period between 1 hour and 1 year stretching between ultradian and infradian rhythms. While not pragmatic to list all possible periods, example circadian rhythm periods are expressly provided with the understanding that relevant periods for ultradian and infradian rhythms are additionally expressly considered as part of the present disclosure. Example circadian rhythm periods include a period between 18 and 30 hours, 18 and 29 hours, 18 and 28 hours, 18 and 27 hours, 18 and 26 hours, 18 and 25 hours, 18 and 24 hours, 19 and 30 hours, 19 and 29 hours, 19 and 28 hours, 19 and 27 hours, 19 and 26 hours, 19 and 25 hours, 19 and 24 hours, 20 and 30 hours, 20 and 29 hours, 20 and 28 hours, 20 and 27 hours, 20 and 26 hours, 20 and 25 hours, 20 and 24 hours, 21 and 30 hours, 21 and 29 hours, 21 and 28 hours, 21 and 27 hours, 21 and 26 hours, 21 and 25 hours, 21 and 24 hours, 22 and 30 hours, 22 and 29 hours, 22 and 28 hours, 22 and 27 hours, 22 and 26 hours, 22 and 25 hours, 22 and 24 hours, 23 and 30 hours, 23 and 29 hours, 23 and 28 hours, 23 and 27 hours, 23 and 26 hours, 23 and 25 hours, 23 and 24 hours, 24 and 30 hours, 24 and 29 hours, 24 and 28 hours, 24 and 27 hours, 24 and 26 hours, and 24 and 25 hours.

In accordance with various embodiments, the biologically defined time-varying temperature profile may comprise a circadian rhythm with an amplitude between 0.5° C. and 3° C., 0.5° C. and 2.5° C., 0.5° C. and 2.0° C., 0.5° C. and 1.5° C., 0.5° C. and 1° C., 1° C. and 3° C., 1° C. and 2.5° C., 1° C. and 2.0° C., 1° C. and 1.5° C., 1.5° C. and 3° C., 1.5° C. and 2.5° C., or 1.5° C. and 2.0° C.

In accordance with various embodiments, the biologically defined time-varying temperature profile may comprise a circadian rhythm with a period and an amplitude formed by the combinations of the various disclosed periods and amplitudes.

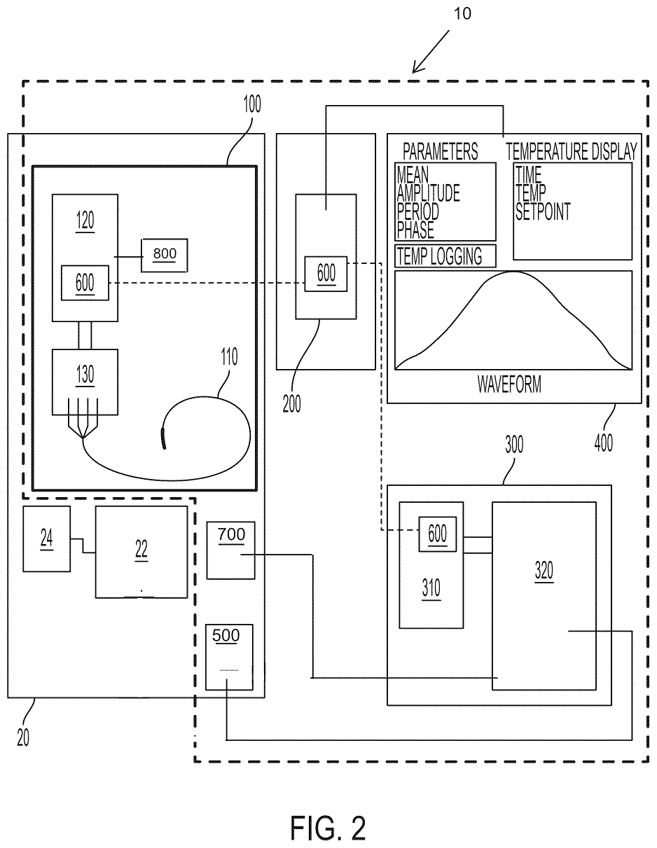

In accordance with one or more embodiments, the programmable waveform engine 400 is communicatively coupled to a graphical user interface (GUI) permitting programming of the time-varying temperature profile and real-time display of temperature parameters. The GUI is designed for managing interactions between the central microcontroller 200 and/or the programmable waveform engine 400 and a user. The GUI may be presented on a display device, such as a monitor, screen, or touchscreen of a computing device (e.g., smartphone, tablet, laptop, desktop, server, or wearable device). The GUI provides visual elements, such as buttons, icons, menus, and text fields, which allow a user to input data, manipulate information, and receive output from the programmable waveform engine 400. In accordance with one or more embodiments, the GUI displays real-time temperature parameters including current measured temperature, target setpoint temperature, elapsed experiment time, and a graphical representation of the programmed waveform, as illustrated in FIGS. 1 and 2. In accordance with one or more embodiments, the GUI further provides user-adjustable waveform parameters including mean temperature, amplitude, period, and phase, allowing a user to define or modify the biologically defined time-varying temperature profile without specialized coding expertise.

A GUI is generically illustrated in FIGS. 1 and 2, but the specific layout and presentation of the GUI is not meant to be limited to the specific embodiment illustrated in FIGS. 1 and 2. Specifically, while FIGS. 1 and 2 show specific layouts and elements, it should be understood that these are exemplary, and the present disclosure is not limited to the specific constructional details shown. Variations, modifications, additions, or omissions may be made to the GUI components, layouts, and display sequences without departing from the scope of the present disclosure. For example, the elements may be rearranged, combined, or separated, and the functionality may be implemented across different screen sizes and operating environments. Further, the GUI components are configured to respond to user interactions (e.g., clicks, touches, swipes, gestures, voice commands). The hardware displaying the GUI and the programmable waveform engine 400 continuously monitors inputs to trigger actions associated with the displayed elements. The displayed information may change dynamically based on the input, and the GUI may present real-time updates based on data processed by the programmable waveform engine 400 and/or central microcontroller 200. In accordance with one or more embodiments, the GUI is further configured to provide data logging and visualization tools enabling a user to review historical temperature log data, export data in formats compatible with standard laboratory analysis software, and monitor experiment progress remotely via a local network connection. In accordance with one or more embodiments, the GUI further provides alert functionality configured to notify a user of temperature deviations exceeding a user-defined threshold, enabling prompt identification and correction of experimental anomalies.

In one or more embodiments, the central microcontroller 200 may be equipped with an accurate real-time clock to ensure that the biologically defined time-varying temperature profile received from the programmable waveform engine 400 can be regulated locally without the need for constant communication with the programmable waveform engine 400. Such arrangement also allows for retained control and retention of the biologically defined time-varying temperature profile in the event of a communication loss with the programmable waveform engine 400.

The power modulation interface 300 comprises a power modulation interface microcontroller 310 configured to modulate electrical power delivered exclusively to the independent internal heating element 500 positioned within the interior chamber of the existing tissue culture incubator 20, without modification of or interference with the internal control circuitry, native heating element 22, or native temperature controller 24 of the existing tissue culture incubator 20. Specifically, in accordance with one or more embodiments, the power modulation interface 300 controls the temperature within the existing tissue culture incubator 20 by regulating power delivery to the independent internal heating element 500 in response to control signals received from the central microcontroller 200. The power modulation interface 300 includes a power relay 320 that selectively modulates electrical power delivered to the independent internal heating element 500. The existing tissue culture incubator 20 is configured with its native temperature controller 24 set to maintain the minimum temperature of the desired oscillation profile, establishing a stable thermal baseline. The independent internal heating element 500 is then modulated by the power modulation interface 300 to superimpose the programmable oscillatory temperature profile onto this stable thermal baseline, thereby achieving the biologically defined time-varying temperature profile within the interior chamber of the existing tissue culture incubator 20. As the native temperature controller 24 is set to the minimum temperature of the desired oscillation profile, the native temperature controller 24 ensures that the minimum temperature is reliably maintained as a thermal baseline even in the event of failure of the power modulation interface 300, providing an inherent safety mechanism that prevents temperatures from falling below the user-defined minimum.

The power modulation interface microcontroller 310 receives instructions from the central microcontroller 200 and adjusts the state of the power relay 320 to effectuate the instructions from the central microcontroller 200. Specifically, the central microcontroller 200 determines if additional heating is required within the existing tissue culture incubator 20 based on various factors such as deviation from the biologically defined time-varying temperature profile or the temperature trend within the existing tissue culture incubator 20. Temperature reduction within the existing tissue culture incubator 20 is typically achieved passively by reducing or ceasing power delivery to the independent internal heating element 500 and allowing the temperature within the interior chamber to passively equilibrate toward the stable thermal baseline maintained by the native temperature controller 24, with the rate of passive thermal equilibration actively controlled by the central microcontroller 200 via the closed feedback loop to conform to the descending phases of the biologically defined time-varying temperature profile. The central microcontroller 200 in one or more embodiments may transmit complete instructions to the power modulation interface microcontroller 310 such that the power modulation interface microcontroller 310 simply generates the appropriate signal to instruct the power relay 320 to modulate power delivery to the independent internal heating element 500. In further embodiments, the central microcontroller 200 may transmit relevant parameters such as deviation from the biologically defined time-varying temperature profile or the temperature trend within the existing tissue culture incubator 20 to the power modulation interface microcontroller 310 which determines an appropriate power delivery modulation scheme before generating a signal to instruct the power relay 320.

Similar to the central microcontroller 200 and the temperature sensing system microcontroller 120, the power modulation interface microcontroller 310 may be any suitable processing device capable of executing instructions to perform the operations described herein, such as a device interchangeably referred to as a microprocessor, a microcontroller, a System-on-a-Chip (SoC), a digital signal processor (DSP), a field-programmable gate array (FPGA), or an application-specific integrated circuit (ASIC). In one embodiment, the power modulation interface microcontroller 310 includes a central processing unit (CPU), a memory (e.g., RAM, ROM, Flash memory), and input/output (I/O) peripherals. The memory may store machine-readable instructions that, when executed by the CPU, cause power modulation interface microcontroller 310 to perform the methods and algorithms described in connection with the power modulation interface microcontroller 310. In accordance with one or more specific embodiments, the power modulation interface microcontroller 310 comprises an ESP32 or equivalent microcontroller configured to receive control signals from the central microcontroller 200 via ESP-NOW protocol and generate appropriate signals to instruct the power relay 320 to modulate power delivery to the independent internal heating element 500. One skilled in the art has familiarity with microcontrollers in general and is capable of selection of an appropriate microcontroller for governing the specific operational guidelines of the power modulation interface microcontroller 310. While a specific microcontroller is described, it should be understood that the techniques described herein are not limited to a particular hardware configuration, and the components may be distributed across multiple devices or integrated into a single component.

In accordance with one or more embodiments, heating within the existing tissue culture incubator 20 is achieved by modulating power delivered to the independent internal heating element 500 positioned within the interior chamber of the existing tissue culture incubator 20, via control of the power relay 320 of the power modulation interface 300. The independent internal heating element 500 is electrically connected to the power relay 320 such that the power relay 320 is capable of modifying the power supplied exclusively to the independent internal heating element 500 without modification of or interference with the native heating element 22 or native temperature controller 24 of the existing tissue culture incubator 20. In one or more embodiments, the power relay 320 wholesale connects and disconnects power to the independent internal heating element 500 such that the independent internal heating element 500 is cycled between full power and no power. Rate of heating may be controlled by alternating between powered and unpowered states of the independent internal heating element 500 to extend or shorten the heating profile to align with the biologically defined time-varying temperature profile. In one or more further embodiments, the power relay 320 may function as a rheostat to provide a continuously variable power output to the independent internal heating element 500. Specifically, the power relay 320 may provide 0%, 10%, 20%, 30%, 40%, 50%, 60%, 70%, 80%, 90%, or 100% of maximal power to the independent internal heating element 500, allowing for rate of heating to be precisely controlled by the temperature oscillation overlay device 10 in accordance with the biologically defined time-varying temperature profile. Temperature reduction within the existing tissue culture incubator 20 is achieved passively by reducing or ceasing power delivery to the independent internal heating element 500 and allowing the temperature within the interior chamber to passively equilibrate toward the stable thermal baseline maintained by the native temperature controller 24, with the rate of passive thermal equilibration actively controlled by the central microcontroller 200 via the closed feedback loop to conform to the descending phases of the biologically defined time-varying temperature profile.

In accordance with one or more embodiments, temperature reduction within the existing tissue culture incubator 20 is achieved passively by reducing or ceasing power delivery to the independent internal heating element 500 via the power relay 320, and allowing the temperature within the interior chamber to naturally equilibrate toward the stable thermal baseline established by the native temperature controller 24, which is set to the minimum temperature of the desired oscillation profile. As the native temperature controller 24 maintains the minimum temperature of the desired oscillation profile as a stable thermal baseline, reducing or ceasing power to the independent internal heating element 500 allows the temperature within the interior chamber to passively decrease toward this baseline without the need for active cooling mechanisms. The rate of passive temperature reduction may be influenced by adjusting the rate at which power delivery to the independent internal heating element 500 is reduced, allowing the temperature oscillation overlay device 10 to achieve smooth temperature transitions consistent with the biologically defined time-varying temperature profile. In one or more embodiments, the power relay 320 may provide continuously variable power output to the independent internal heating element 500, specifically providing 0%, 10%, 20%, 30%, 40%, 50%, 60%, 70%, 80%, 90%, or 100% of maximal power, allowing for precise control of the rate of passive temperature reduction by the temperature oscillation overlay device 10. Such variable power control allows for a steady-state low power to be applied to the independent internal heating element 500**, wherein the central microcontroller 200 actively monitors the measured temperature via the closed feedback loop and adjusts power delivery to the independent internal heating element 500 to control the rate of passive thermal equilibration, thereby achieving smooth temperature transitions that conform to the descending phases of the biologically defined time-varying temperature profile at all times.

In one or more embodiments, the power relay 320 comprises a default condition of no power delivery to the independent internal heating element 500. By configuring the power relay 320 to default to no power delivery to the independent internal heating element 500, it is ensured the independent internal heating element 500 will not generate heat even when the device 10 is not functioning. In a failure state, the native temperature controller 24 retains full and uninterrupted control of the temperature of the existing tissue culture incubator 20, effectively converting the existing tissue culture incubator 20 back to its standard static temperature state as if the temperature oscillation overlay device 10 were not installed, with the temperature stabilizing at the minimum temperature of the desired oscillation profile as set by the native temperature controller 24. Specifically, in such a failure state the native temperature controller 24 maintains full control of the temperature of the existing tissue culture incubator 20 and effectively converts the existing tissue culture incubator 20 back to the state prior to without the temperature oscillation overlay device 10 being installed.

In various embodiments, the central microcontroller 200 maintains temperature within the tissue culture incubator 20 within +0.3° C., +0.25° C., +0.2° C., +0.15° C., or +0.1° C. of a programmed setpoint as determined by the time-varying temperature profile. In accordance with one or more embodiments, this precision is achieved across the full operating range of 30° C. to 50° C. and is maintained continuously throughout the duration of the experiment, including during long-term experiments of 10 days or more. In accordance with one or more specific embodiments, the current prototype of the temperature oscillation overlay device 10 achieves a precision of approximately +0.3° C., with the next-generation prototype targeting a precision of approximately +0.2° C. through incorporation of the independent internal heating element 500 and closed feedback loop control described herein

In one or more embodiments and with reference to FIG. 2, the temperature oscillation overlay 10 may further comprise an auxiliary temperature control unit 700 to augment heating from the native heating element 22 and/or induce cooling. The auxiliary control unit 700 provides additional heating capacity where required, for example to achieve more rapid temperature increases or more dramatic temperature amplitudes consistent with the biologically defined time-varying temperature profile. In one or more embodiments, temperature reduction remains achieved passively by reducing or ceasing power delivery to the independent internal heating element 500 and/or the auxiliary temperature control unit 700, where power delivery is reduced or ceased and the rate of passive thermal equilibration is controlled to conform to the programmed waveform. It In further embodiment, the auxiliary temperature control unit 700 positioned within the interior chamber of the existing tissue culture incubator 20 also allows for forced cooling to occur removing reliance on atmospheric cooling of the existing tissue culture incubator 20 while conforming to the biologically defined time-varying temperature profile. The auxiliary temperature control unit 700 may be under direct control of the central microcontroller 200 or may receive instruction from the power modulation interface 300 and more specifically the power modulation interface microcontroller 310. Multiple auxiliary temperature control units 700 may be additively utilized to provide increased heating capacity for quicker and/or more dramatic temperature transitions consistent with the biologically defined time-varying temperature profile.

In accordance with one or more embodiments, the auxiliary temperature control unit 700 may be a thermoelectric cooling/heating mechanism. A thermoelectric cooling/heating mechanism utilizes the Peltier effect to heat and/or cool. Specifically, a thermoelectric cooling/heating mechanism uses DC electricity to transfer heat across a semiconductor module, creating a hot side and a cold side without moving parts or refrigerants. By reversing the current, the module reverses, enabling, and allowing for precise, compact temperature control.

In accordance with one or more embodiments, the auxiliary temperature control unit 700 may utilize forced air convention for heating and/or cooling. Forced air convection may be utilized to heat or cool the tissue culture incubator 20 by using a furnace, heat pump, or air conditioner to heat/cool air which is passed through a blower fan into and/or around the tissue culture incubator 20. This active circulation continuously draws in, treats, and redistributes air, enabling fast, even temperature distribution compared to natural convection.

In accordance with one or more embodiments, the auxiliary temperature control unit 700 may utilize resistive heating. In effect, the native heating element 22 may be duplicated with the auxiliary temperature control unit 700 to provide accessory heating to that able to be provided by the native heating element 22.

In one or more embodiments, the temperature oscillation overlay device 10 further comprises one or more data storage modules 800 communicatively coupled to the temperature sensing system microcontroller 120. The data storage module 800 is configured to store timestamped temperature data from the one or more temperature sensors 110. Retention of the timestamped temperature data allows for logging and review of temperatures within the tissue culture incubator 20. Additionally, in the event of loss of communication between the temperature sensing system 100 and the central microcontroller 200, the timestamped temperature data may be retained and then transmitted upon establishment of communicative connectivity. Further, the timestamped temperature data also allows multiple temperature readings from the at least one temperature sensor 110 to be obtained and temporarily stored before transmission to the central microcontroller 200 as a single data burst to allow for one or more of the wireless communication modules 600 operatively coupled to the central microcontroller 200, the temperature sensing system microcontroller 120, and the power modulation interface microcontroller 310 to be powered off or into a low-power state for energy conservation.

In one or more embodiments, the data storage module 800 is a compact, non-volatile flash memory card such as an SD card. In further embodiments, the data storage module 800 comprises a solid-state data storage device with USB or other standardized connectivity to the temperature sensing system microcontroller 120.

In one or more embodiments, the temperature oscillation overlay device 10 further comprises a plurality of wireless communication modules 600. The wireless communication modules are operatively coupled to the central microcontroller 200, the temperature sensing system microcontroller 120, and the power modulation interface microcontroller 310 to effectuate wireless communication between the temperature sensing system microcontroller 120 and the central microcontroller 200 and between the central microcontroller 200 and the power modulation interface microcontroller 310. Wireless communication between the various microcontrollers of the temperature oscillation overlay device 10 allows for disparate placement of the tissue culture incubators 20 and the central microprocessor 200 and/or the programmable waveform engine 400 and/or associated GUI. In one or more embodiments, the wireless communication modules 600 may be configured to allow wireless transmission of data stored in the one or more data storage modules 800. The wireless communication modules 600 may operate using any known wireless data transfer protocol. In accordance with one or more embodiments, the wireless communication modules 600 operate using a peer-to-peer protocol configured for direct device-to-device communication without reliance on external network infrastructure. In one or more specific embodiment, the wireless communication modules 600 operate using ESP-NOW (Espressif Systems, Shanghai, China) which is a fast, connectionless, and low-power wireless communication which enables direct device-to-device communication without a router. ESP-NOW is well suited for the temperature oscillation overlay device 10 as such protocol is ideal for sending small packets of up to 250 bytes with a high connectivity reliability of greater than 99%. Further, non-limiting examples of wireless data transfer protocols which may be utilized in accordance with various embodiments include Bluetooth (IEEE 802.15.1) and Wi-Fi (IEEE 802.11).

In one or more embodiments, the central microprocessor 200 provides real-time remote monitoring and alert notification upon deviation from programmed temperature parameters. In accordance with various embodiments, the alert notifications may be displayed on the graphical user interface, provided as a visual or auditory alarm, or transmitted to an external device for receipt by a user. For example, a visual alarm may be provided via a flashing or strobing light connected directly or wirelessly to the temperature oscillation overlay device 10, an auditory alarm may be provided via a speaker or bell connected directly or wirelessly to the temperature oscillation overlay device 10, or a message may be conveyed to a user via email or text message alerting the user to a temperature anomaly. Each of these solutions may be implemented in accordance with known protocols and are developmentally within the ability of one skilled in the art without requiring undue development or experimentation.

In accordance with various embodiments, the alert notification upon deviation from programmed temperature parameters may be triggered based on one or more of a different types of deviation from the programmed temperature parameters. For example, deviation by a magnitude may trigger an immediate alert or deviation by a lesser magnitude for a threshold time period may trigger a delayed alert. In one or more embodiments, the alert notification may include indication of the cause of the alert and/or include or display temperature data for the user to diagnose the severity of the alert. The temperature data provided with an alert notification may include current and/or historical temperatures of the existing tissue culture incubator 20 as well as current and/or historical target temperatures based on the biologically defined time-varying temperature profile.

In accordance with one or more embodiments, the central controller 200 is configured to receive temperature measurements from a plurality of tissue culture incubators 20 and relay instructions to a plurality of power modulation interfaces 300 associated with the plurality of tissue culture incubators 20. Specifically, a single central controller 200 and associated programmable waveform engine 400 may manipulate the temperature of more than one tissue culture incubator 20. Specifically, the central microcontroller 200 may be configured to coordinate biologically defined time-varying temperature profiles across the plurality of tissue culture incubators 20 simultaneously, enabling experimental designs requiring phase-shifted or otherwise coordinated temperature oscillations across multiple independent cell culture systems. In accordance with one or more further embodiments, the central microcontroller 200 is configured to monitor temperature measurements from each of the plurality of tissue culture incubators 20 independently and generate alert notifications via the graphical user interface upon detection of temperature deviations exceeding a user-defined threshold in any of the plurality of tissue culture incubators 20.

Further, as academic and commercial research laboratories commonly include multiple tissue culture incubators 20, operating more than one tissue culture incubator 20 with a single central controller 200 reduces unnecessary duplication of components and reduces overall cost. Further, as a laboratory acquires and/or replaces tissue culture incubators 20 they may be integrated into the temperature oscillation overlay device 10 without requiring acquisition of an entirely new system.

Having described the temperature oscillation overlay device 10 and its operation, methods which leverage the temperature oscillation overlay device 10 and its capabilities are discussed.

The temperature oscillation overlay device 10 may be leveraged to entrain circadian gene expression in cultured cells. Specifically, in accordance with one or more embodiments, a method of entraining circadian gene expression in cultured cells is provided. The method generally comprises maintaining the cells in a standard tissue culture incubator 20; superimposing using the temperature oscillation overlay device 20 a continuous oscillatory temperature profile having a period of 20 to 28 hours and an amplitude between 0.5° C. and 3° C.; and maintaining the oscillatory temperature profile for at least five consecutive circadian cycles. Accordingly, the oscillatory temperature profile functions as a sustained zeitgeber that increases circadian amplitude and reduces inter-replicate variability relative to serum shock synchronization.

In accordance with various embodiments, the method of entraining circadian gene expression in cultured cells utilizes mammalian, insects, fish, reptile, amphibian, birds, bacteria, or fungi cells. Each of these cell types are from species which exhibit circadian biological clocks. While mean temperature, amplitude, period, and other parameters may vary between various species, each exhibit a circadian rhythm. In one or more specific embodiments, the cultured cells are mammalian.

In accordance with various embodiments, the oscillatory temperature profile has an amplitude of ±0.5° C. to 3° C., ±0.5° C. to 2.5° C., ±0.5° C. to 2.0° C., ±0.5° C. to 1.5° C., ±0.5° C. to 1° C., ±1° C. to 3° C., ±1° C. to 2.5° C., ±1° C. to 2.0° C., ±1° C. to 1.5° C., ±1.5° C. to 3° C., ±1.5° C. to 2.5° C., or ±1.5° C. to 2.0° C. relative to a mean temperature of 35° C., 36° C., 37° C., 38° C., or 39° C. In one or more specific embodiments, the oscillatory temperature profile has an amplitude of ±1 to 2° C. relative to a mean temperature of 37° C.

In accordance with one or more embodiments, the oscillatory temperature profile is maintained for at least 5 days, at least 6 days, at least 7 days, at least 8 days, at least 9 days, at least 10 days, at least 11 days, at least 12, days at least 13, days, at least 14 days, at least 21 days, or at least 28 days. It will be appreciated that extending the period the oscillatory temperature profile is maintained allows for extended research activities as well as maintaining the cultured cells in greater alignment with in vivo compared to variability introduced with reliance on serum shock synchronization where circadian markers are initially present but quickly fade.

The temperature oscillation overlay device 10 may also be leveraged to detecting circadian regulatory elements in a gene promoter. Specifically, in accordance with one or more embodiments, a method of detecting circadian regulatory elements in a gene promoter is provided. The method generally comprises introducing into cultured cells a reporter construct comprising a promoter region operatively linked to a reporter gene; maintaining the cultured cells in a standard tissue culture incubator; superimposing using the temperature oscillation overlay device of claim 1 a continuous oscillatory temperature profile having a period of approximately 24 hours, including 18 to 30 hours, and an amplitude between 0.5° C. and 3° C.; maintaining the oscillatory temperature profile for at least five consecutive circadian cycles, and measuring reporter gene expression over time. Accordingly, functional differences in promoter activity under the superimposed continuous oscillatory temperature profile identify the presence or functional contribution of circadian regulatory elements.

In accordance with various embodiments, the detecting circadian regulatory elements in a gene promoter utilizes mammalian, insects, fish, reptile, amphibian, birds, or fungi cells. Each of these cell types are from species which exhibit circadian biological clocks. While mean temperature, amplitude, period, and other parameters may vary between various species, each exhibit a circadian rhythm. In one or more specific embodiments, the cultured cells are mammalian.

In accordance with one or more embodiments, the oscillatory temperature profile is maintained for at least 5 days, at least 6 days, at least 7 days, at least 8 days, at least 9 days, at least 10 days, at least 11 days, at least 12, days at least 13, days, at least 14 days, at least 21 days, or at least 28 days. It will be appreciated that extending the period the oscillatory temperature profile is maintained allows for extended research activities as well as maintaining the cultured cells in greater alignment with in vivo compared to variability introduced with reliance on serum shock synchronization where circadian markers are initially present but quickly fade.

In accordance with one or more embodiments, the reporter gene comprises a luciferase gene. Although luciferase is used in the examples described here, any reporter gene that produces a detectable signal can be used. This includes bioluminescent reporters (such as firefly luciferase, Renilla luciferase, or NanoLuc), fluorescent proteins (such as GFP or mCherry), and enzymatic reporters (such as β-galactosidase). Specifically, in accordance with one or more specific embodiments, the luciferase gene comprises a firefly luciferase gene, a Renilla luciferase gene, or a NanoLuc luciferase gene operatively linked to the promoter region under study. In accordance with one or more further embodiments, the reporter gene is not limited to luciferase and may comprise any reporter gene capable of producing a detectable signal in response to promoter activity, including but not limited to fluorescent protein reporters such as GFP and mCherry and variants thereof, and enzymatic reporters such as beta-galactosidase and alkaline phosphatase. In accordance with one or more further embodiments, the luciferase reporter gene enables real-time continuous monitoring of promoter activity via bioluminescence detection, providing a temporal resolution sufficient to detect circadian oscillations in reporter gene expression over experimental periods of 10 days or more without the need for cell fixation or other disruptive sampling methods. The specific reporter gene used is not central to the invention, which is defined by the method of applying programmable temperature oscillations to the cell culture system rather than by the means of detecting the biological response.

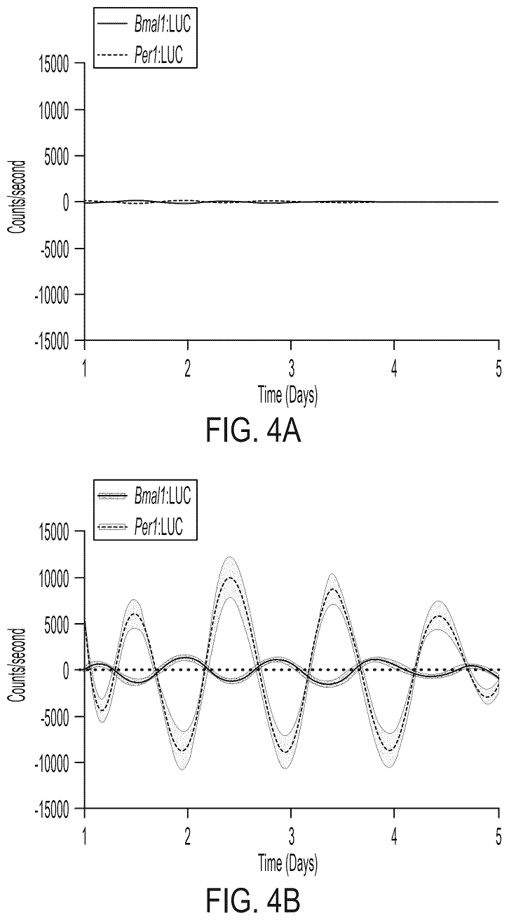

In accordance with one or more embodiments, the circadian regulatory element comprises an E-box sequence. An E-box (enhancer box) is a short, conserved DNA regulatory motif. Specifically, the short, conserved DNA regulatory motif is the palindromic sequence 5′-CANNTG-3′ (where Nis any nucleotide), found in the promoter or enhancer regions of eukaryotic genes. The most common E-box sequence is 5′-CACGTG-3′. The E-box sequence acts as a protein-binding site for basic helix-loop-helix (bHLH) transcription factors, controlling gene expression in development, metabolism, and circadian rhythms. The ChronoCulture device improves the detection of E-box activity because it produces stronger, more stable circadian oscillations in cultured cells compared to traditional serum shock synchronization. As shown in FIG. 4, when cells were maintained under temperature oscillation conditions, E-box driven promoter activity was robust and clearly measurable. When the E-box sequence was deleted from the hGJA1 promoter, the change in bioluminescent signal was large and statistically significant, something that would be difficult or impossible to detect reliably under serum shock conditions due to the low amplitude and rapid dampening of rhythms. In other words, the device makes E-box activity visible and measurable in experiments where it would otherwise be too weak to detect.

In accordance with one or more further embodiments, the circadian regulatory element is not limited to E-box sequences and may comprise any transcription factor binding site or other regulatory element whose activity is modulated in a circadian phase-dependent manner, including but not limited to D-box sequences, RRE sequences, and other clock-controlled regulatory elements.

In accordance with one or more further embodiments, functional differences in circadian regulatory element activity are identified by comparing reporter gene expression amplitude, period, acrophase, or baseline expression between a promoter construct comprising the intact regulatory element and a promoter construct comprising a deletion or mutation of the regulatory element.

### Examples

The temperature oscillation overlay device 10 and the disclosed methods were experimentally demonstrated.

As previously discussed, temperature is a fundamental regulator of biological processes, influencing everything from enzyme kinetics and protein folding to gene expression and cellular metabolism. In living organisms, temperature fluctuations follow circadian rhythms, which are critical in maintaining physiological homeostasis and synchronizing biological clocks. These temperature oscillations impact various physiological outputs, including heart rate, metabolic rate, blood pressure, hormone secretion, sleep-wake cycles, cognitive performance, immune function2, and gene expression patterns. At the cellular level, temperature rhythms affect protein synthesis rates, enzyme activity, and the timing of cell division. Despite its profound importance, the dynamic nature of temperature in biological systems has been largely not accounted for in traditional tissue culture techniques, which typically maintain a constant temperature or are manually switched between two temperatures. This oversight represents a significant gap between in vivo conditions and in vitro models, potentially limiting the physiological relevance of cell culture experiments. This limitation is particularly consequential in chronobiology, where the dampening of circadian oscillations under static temperature conditions results in low-amplitude circadian gene expression, high inter-replicate variability, and rapid loss of rhythm persistence within 3 to 5 days, rendering traditional synchronization methods such as serum shock insufficient for long-term circadian studies. Furthermore, the inability to replicate physiologically relevant temperature dynamics in vitro has limited the utility of cell culture models in drug development, where circadian timing is increasingly recognized as a critical variable in drug efficacy and toxicity, and in organoid and tissue engineering applications, where dynamic temperature environments more faithfully replicate the in vivo conditions under which complex tissue architecture and function develop.

Accordingly, the present disclosure introduces the ability to apply programmable temperature oscillations into tissue culture systems to more accurately mimic the natural cellular environment opening avenues to revolutionize experimental approaches to cell biology, chronobiology, cardiac physiology, immunology and drug discovery. Specifically, the temperature oscillation overlay device 10 helps to bridge the gap between laboratory conditions and real-world biological processes, opening new avenues for research and therapeutic development. In accordance with the biological validation data described herein, the temperature oscillation overlay device 10 demonstrates greater than 15-fold improvement in circadian amplitude for Bmal1 reporter constructs and greater than 95-fold improvement for Per1 reporter constructs compared to traditional serum shock synchronization, with rhythm persistence extended from 3 to 5 days under serum shock conditions to 10 to 15 days under temperature oscillation conditions.

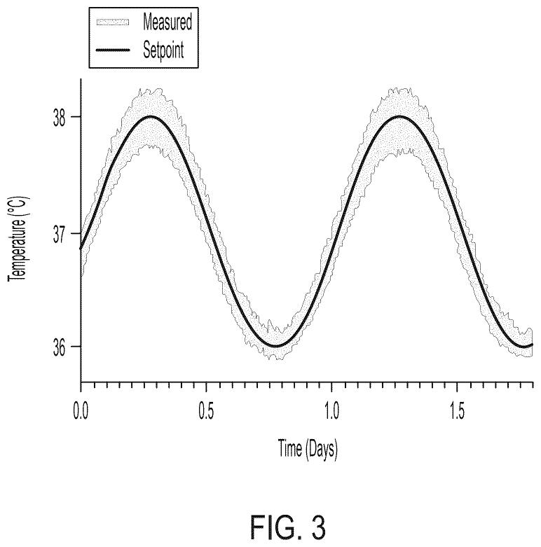

To demonstrate the potential of temperature oscillations in tissue culture experiments, the temperature oscillation overlay device 10 was utilized to introduce cyclic heating within a tissue culture incubator. Specifically, the native temperature controller 24 of the existing tissue culture incubator 20 was set to 36° C. to establish a stable thermal baseline representing the minimum temperature of the desired oscillation profile, and the independent internal heating element 500 was modulated by the power modulation interface 300 under closed feedback loop control of the central microcontroller 200 to superimpose a sinusoidal oscillatory temperature profile onto this baseline. The incubator temperature was cyclically varied between 36° C. and 38° C. with a 24 hour period, representing a physiologically relevant circadian temperature oscillation of 1° C. amplitude centered on a mean temperature of 37° C. With reference to FIG. 3, the cyclical heating profile illustrates the measured temperature was maintained near the setpoint temperature over the experimental timeline demonstrating the ability of the temperature oscillation overlay device 10 to achieve precise, reproducible temperature oscillations with a precision of approximately +0.3° C. throughout the experimental period. This cyclic heating pattern continued and was repeated for each experiment day with temperature data recorded and logged. The temperature data was continuously recorded and stored both wirelessly via ESP-NOW protocol and locally via SD card storage, providing redundant data backup independent of network connectivity.

Building on this understanding of the influence of temperature on cellular processes, experimentation was completed to explore how specific circadian clock genes respond to temperature cues and regulate biological rhythms at the molecular level. The core circadian clock feedback loop involves the basic helix-loop-helix transcription factors BMAL1 and CLOCK (positive limb of the circadian clock), which enhance the transcription of Period (Per) and Cryptochrome (Cry) genes. PER1 and CRY proteins then inhibit BMAL1 and CLOCK activity, forming the opposing limb. As PER1 and CRY proteins degrade, inhibition of BMAL1 and CLOCK decreases, allowing the cycle to restart. Components of the positive and negative limbs of the core circadian clock mechanism are expressed in an antiphase pattern. This molecular feedback loop generates self-sustained oscillations with a period of approximately 24 hours, which can be entrained by external time cues known as zeitgebers, including light, feeding, and temperature. The temperature oscillation overlay device 10 exploits temperature as a continuous zeitgeber to synchronize and maintain circadian gene expression rhythms in cultured cells, providing a physiologically relevant and sustained synchronization stimulus that overcomes the limitations of traditional one-time synchronization methods such as serum shock. The temperature oscillation overlay device 10 was used in conjunction with reporter constructs comprising circadian gene promoters operatively linked to a reporter gene, specifically a luciferase reporter gene for the present testing, to enable real-time monitoring of circadian gene expression dynamics in cultured cells over extended experimental periods of up to 10 days or more.

Demonstration of the increased expression of the positive and negative limbs of the core circadian clock mechanism was completed. Specifically, real-time bioluminescence (LumiCycle, Actimetrics) was used to investigate the circadian expression of cloned promoter-reporter constructs. Traditional methods employ serum shock (50% serum) or dexamethasone to align the clocks of individual cells in the culture dish before Lumicycle placement. To demonstrate the effect of temperature cycling, experimentation was completed using traditional methods of serum shock with 50 percent horse serum (Atlanta Biologicals) as well as using the method and the temperature oscillation overlay device 10 of the present disclosure. With reference to FIG. 4A, Lumicycle data recorded from C2C12 myotubes that were transiently transfected with the promoter luciferase constructs for Bmal1 and Per1 following serum shock with 50 percent horse serum (Atlanta Biologicals) is illustrated. Similarly, with reference to FIG. 4B, Lumicycle data recorded from C2C12 myotubes that were transiently transfected with the promoter luciferase constructs for Bmal1 and Per1 and then temperature cycled in conformity with the temperature profile illustrated in FIG. 3 is illustrated. It is noted that Bmal1 and Per1 were selected for analysis as they represent opposing limbs of the core circadian clock mechanism. Bioluminescence data was recorded at 10-minute intervals for at least 5 days, and the data was analyzed for circadian parameters, including period, phase, and amplitude, with Lumicycle software.

A visual review of FIGS. 4A and 4B plainly illustrates the substantially increased amplitude for both Bmal1 and Per1 with temperature oscillation. Specifically, temperature oscillation conditions produced a greater than 15-fold increase in Bmal1:LUC amplitude and a greater than 95-fold increase in Per1:LUC amplitude compared to serum shock synchronization. Furthermore, inter-replicate variability in both period length and acrophase timing was substantially reduced under temperature oscillation conditions, with period standard deviation reduced by 38% for Bmal1:LUC and 89% for Per1:LUC, and acrophase standard deviation reduced by 87% for Bmal1:LUC and 92% for Per1:LUC compared to serum shock. Baseline expression levels were also substantially elevated under temperature oscillation conditions, with Bmal1:LUC baseline expression increased by approximately 380% and Per1:LUC baseline expression increased by approximately 1,100% compared to serum shock conditions.

The full complement of collected data, including amplitude, rhythm persistence, period standard deviation, acrophase standard deviation, and expression is provided in Table 1.

TABLE 1

| | | Temperature | |
|---|---|---|---|
| Parameter | Serum Shock | Oscillation | Improvement |
| Bmal1:LUC | 100.4 ± 40.52 | 1,602 ± 264.5 | >15-fold |
| amplitude | | | |
| (counts/sec) | | | |
| Per1:LUC | 106.1 ± 28.31 | 10,199 ± 4,133 | >95-fold |
| amplitude | | | |
| (counts/sec) | | | |
| Rhythm | 3-5 days | 10-15 days | 2-5x longer |
| persistence | | | |
| Bmal1:LUC | 1.22 | 0.75 | 38% reduction |
| period SD | | | |
| Per1:LUC | 1.20 | 0.13 | 89% reduction |
| period SD | | | |
| Bmal1:LUC | 3.47 | 0.44 | 87% reduction |
| acrophase SD | | | |
| Per1:LUC | 5.02 | 0.42 | 92% reduction |
| acrophase SD | | | |
| Bmal1:LUC | Baseline | +380% | Significant |
| baseline | | | elevation |
| expression | | | |
| Per1:LUC | Baseline | +1,100% | Significant |
| baseline | | | elevation |
| expression | | | |

The results show a marked increase in oscillation amplitude for both Bmal1 and Per1 with temperature oscillation. Notably, Bmal1 and Per1 exhibit antiphase oscillation patterns. Moreover, these rhythms persist significantly longer (10-15 days) under programmed temperature oscillations according to the present disclosure without damping compared to traditional methods (3-5 days) which utilized serum shock. This extended rhythm persistence represents a 2 to 5-fold improvement in experimental window compared to serum shock and is particularly significant for long-term chronobiological studies, drug chronopharmacology experiments, and other applications requiring sustained circadian synchronization over multiple days. The combination of substantially increased amplitude, reduced inter-replicate variability, elevated baseline expression, and extended rhythm persistence collectively demonstrates that the temperature oscillation overlay device 10 provides a physiologically superior and experimentally more reproducible method of circadian synchronization in cultured cells compared to traditional serum shock synchronization.

The temperature oscillations providing a strong and consistent zeitgeber enable rhythms to persist with minimal damping over a longer time frame along with the increase in amplitude of expression observed with oscillating temperature enhances the ability to detect and characterize rhythms that might be subtle or undetectable with traditional synchronization methods like serum shock or dexamethasone treatment. For instance, the human GJA1 luciferase promoter reporter typically exhibits very low amplitude oscillations when synchronized by serum shock (data not shown). However, with temperature oscillation, a significant increase in amplitude may be observed. In one experiment, the amplitude increased by approximately 59% (2.73±0.26 for serum shock vs. 4.33±0.52 for temperature oscillation; mean±SEM; n=5-15, p=0.041). This finding demonstrates that the temperature oscillation overlay device 10 enables detection of circadian regulatory activity in gene promoters that would otherwise be undetectable or unreliably measured under traditional synchronization conditions, thereby expanding the range of genes and promoter elements accessible to circadian research. The hGJA1 gene encodes connexin43, a gap junction protein with important roles in cardiac conduction, cell-cell communication, and tissue homeostasis, and its circadian regulation may have significant implications for understanding cardiac arrhythmias and other diseases with known circadian components.

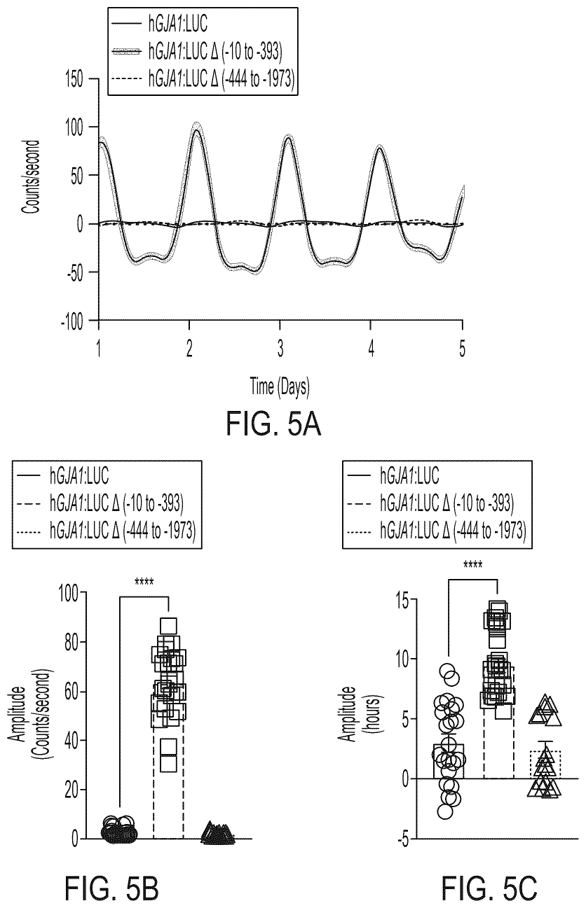

Human GJA1 promoter reporters (control and deletion mutants) show a circadian pattern when transiently transfected in C2C12 myotubes. Deletion of a conserved tandem E-box, from −10 to −393 upstream of the transcriptional start site in the hGJA1 promoter significantly increased the bioluminescent amplitude and shifted the acrophase, as illustrated in FIGS. 5A, 5B, and 5C. Comparatively, deletion of a conserved tandem E-box, from −444 to −1973 upstream of the transcriptional start site in the hGJA1 promoter did not increase the bioluminescent amplitude nor appreciably shift the acrophase. FIGS. 5A, 5B, and 5C illustrate the hGJA1:LUC data under temperature oscillation. Accordingly, in view of the enhanced bioluminescent, it is demonstrated that detecting circadian regulatory elements in a gene promoter is enhanced with the temperature oscillations in accordance with the present disclosure.

Having described various embodiments, it should be understood that the various aspects of the temperature oscillation overlay device configured for removable integration with an existing tissue culture incubator and methods which use the same are described and such aspects may be utilized in conjunction with various other aspects.

In a first aspect, the disclosure provides a temperature oscillation overlay device configured for removable integration with an existing tissue culture incubator having a native temperature controller and a native heating element. The device comprises a temperature sensing system physically independent of the native temperature controller, the temperature sensing system comprising at least one temperature sensor configured for placement within an interior chamber of the incubator and a temperature sensing system microcontroller configured to receive data from the temperature sensor; a central microcontroller configured to receive temperature measurements obtained by the at least one temperature sensor from the temperature sensing system microcontroller; an independent internal heating element configured for placement within the interior chamber of the incubator and operated entirely separately from and without modification of the native heating element or native temperature controller; a power modulation interface comprising a power modulation interface microcontroller configured to selectively modulate electrical power delivered exclusively to the independent internal heating element without modification of the incubator's internal control circuitry; and a programmable waveform engine executed by the central microcontroller and configured to generate a biologically defined time-varying temperature profile; wherein the central microcontroller controls power delivery to the independent internal heating element in a closed feedback loop independent of the native temperature controller to superimpose the oscillatory temperature profile onto a stable thermal baseline established by setting the native temperature controller to the minimum temperature of the desired oscillation profile.

In a second aspect, the disclosure provides the device of the first aspect, in which the biologically defined time-varying temperature profile is representative of a circadian rhythm, an ultradian rhythm, an infradian rhythm, a fever profile, or a hypothermic profile.

In a third aspect, the disclosure provides the device of the first or second aspect, in which the programmable waveform engine permits user-defined adjustment of amplitude, period, phase, and mean temperature of the biologically defined time-varying temperature profile.

In a fourth aspect, the disclosure provides the device of any of the first through third aspects, in which the programmable waveform engine further permits user-defined selection of waveform shape, including sinusoidal, square wave, ramp, and fully custom-defined profiles, and is configured to coordinate temperature profiles across multiple tissue culture incubators simultaneously.

In a fifth aspect, the disclosure provides the device of any of the first through fourth aspects, in which the biologically defined time-varying temperature profile comprises a circadian rhythm with a period between 18 and 30 hours and an amplitude between 0.5° C. and 3° C.

In a sixth aspect, the disclosure provides the device of any of the first through fifth aspects, in which the circadian rhythm temperature profile comprises a sinusoidal waveform centered on a mean temperature within the operating range of 30° C. to 50° C., or more specifically approximately 37° C. in some aspects.

In a seventh aspect, the disclosure provides the device of any of the first through sixth aspects, in which the device further comprises a plurality of wireless communication modules operatively coupled to the central microcontroller, the temperature sensing system microcontroller, and the power modulation interface microcontroller to effectuate wireless communication between the temperature sensing system microcontroller and the central microcontroller and between the central microcontroller and the power modulation interface microcontroller.

In an eighth aspect, the disclosure provides the device of the seventh aspect, in which the wireless communication modules operate using a peer-to-peer protocol configured for direct device-to-device communication without reliance on external network infrastructure.

In a ninth aspect, the disclosure provides the device of the method of any of the first through eighth aspects, in which the device further comprises one or more data storage modules communicatively coupled to the temperature sensing system microcontroller, wherein the data storage module is configured to store timestamped temperature data independent of wireless connectivity.

In a tenth aspect, the disclosure provides the device of any of the first through ninth aspects, in which the at least one temperature sensor comprises a platinum resistance temperature detector.

In an eleventh aspect, the disclosure provides the device of any of the first through tenth aspects, in which the central microcontroller maintains temperature within the tissue culture incubator within +0.3° C., more preferably +0.2° C., of a programmed setpoint as determined by the time-varying temperature profile and in one or more specific aspect this precision is maintained continuously across the full operating range of 30° C. to 50° C. and throughout long-term experiments of 10 days or more.

In a twelfth aspect, the disclosure provides the device of any of the first through eleventh aspects, in which the programmable waveform engine is communicatively coupled to a graphical user interface permitting programming of the time-varying temperature profile and real-time display of temperature parameters.

In a thirteenth aspect, the disclosure provides the device of any of the first through twelfth aspects, in which the central controller is configured to receive temperature measurements from a plurality of tissue culture incubators and relay instructions to a plurality of power modulation interfaces associated with the plurality of tissue culture incubators to modulate power delivery to a plurality of independent internal heating elements.

In a fourteenth aspect, the disclosure provides the device of any of the first through thirteenth aspects, in which wherein the central microprocessor provides real-time remote monitoring and alert notification upon deviation from programmed temperature parameters.

In a fifteenth aspect, the disclosure provides the device of any of the first through fourteenth aspects, in which the device further comprises an auxiliary temperature control unit under control of the central microcontroller to augment heating from the independent internal heating element.

In a sixteenth aspect, the disclosure provides a method of entraining circadian gene expression in cultured cells. The method comprises maintaining the cells in a standard tissue culture incubator; superimposing a continuous oscillatory temperature profile having a period of approximately 24 hours, including periods ranging from 18 to 30 hours and an amplitude between 0.5° C. and 3° C. using the temperature oscillation overlay device of any of the first through thirteenth aspects; and maintaining the oscillatory temperature profile for at least five consecutive circadian cycles, wherein the oscillatory temperature profile functions as a sustained zeitgeber that increases circadian amplitude and reduces inter-replicate variability relative to serum shock synchronization.

In a seventeenth aspect, the disclosure provides the method of the sixteenth aspect, in which the cultured cells are mammalian.

In an eighteenth aspect, the disclosure provides the method of the sixteenth or seventeenth aspect, in which the oscillatory temperature profile has an amplitude of +1 to 2° C. relative to a mean temperature of 37° C.

In a nineteenth aspect, the disclosure provides the method of any of the sixteenth through eighteenth aspects, in which the oscillatory temperature profile is maintained for at least ten consecutive circadian cycles.

In a twentieth aspect, the disclosure provides a method of detecting circadian regulatory elements in a gene promoter. The method comprises introducing into cultured cells a reporter construct comprising a promoter region operatively linked to a reporter gene; maintaining the cultured cells in a standard tissue culture incubator; superimposing a continuous oscillatory temperature profile having a period of approximately 24 hours, including periods ranging from 18 to 30 hours and an amplitude between 0.5° C. and 3° C. using the temperature oscillation overlay device of any of the first through fourteenth aspects; maintaining the oscillatory temperature profile for at least five consecutive circadian cycles, and measuring reporter gene expression over time, wherein differences in amplitude, period, acrophase, or baseline expression of reporter gene activity under the superimposed continuous oscillatory temperature profile identify the presence or functional contribution of circadian regulatory elements.

In a twenty-first aspect, the disclosure provides the method of the twentieth aspect, in which the reporter gene comprises a luciferase gene, a fluorescent protein reporter gene, or an enzymatic reporter gene.

In a twenty-second aspect, the disclosure provides the method of the twentieth or twenty-first aspect, in which the circadian regulatory element comprises an E-box sequence, a D-box sequence, or an RRE sequence.

It should be apparent to those skilled in the art that various modifications and variations may be made to the embodiments described within without departing from the spirit and scope of the claimed subject matter. Thus, it is intended that the specification cover the modifications and variations of the various embodiments described within provided such modification and variations come within the scope of the appended claims and their equivalents.

As used throughout, the singular forms “a,” “an” and “the” include plural referents unless the context clearly dictates otherwise. Thus, for example, reference to “a” component includes aspects having two or more such components, unless the context clearly indicates otherwise.

Optional or optionally means that the subsequently described event or circumstances may or may not occur. The description includes instances where the event or circumstance occurs and instances where it does not occur.

It should be understood that any two quantitative values assigned to a property or measurement may constitute a range of that property or measurement, and all combinations of ranges formed from all stated quantitative values of a given property or measurement are contemplated in this disclosure.

As used here and in the appended claims, the words “comprise,” “has,” and “include” and all grammatical variations thereof are each intended to have an open, non-limiting meaning that does not exclude additional elements or steps.

Having described the subject matter of the present disclosure in detail and by reference to specific embodiments thereof, it is noted that the various details disclosed within should not be taken to imply that these details relate to elements that are essential components of the various embodiments described within, even in cases where a particular element is illustrated in each of the drawings that accompany the present description. Further, it should be apparent that modifications and variations are possible without departing from the scope of the present disclosure, including, but not limited to, embodiments defined in the appended claims. More specifically, although some aspects of the present disclosure are identified as particularly advantageous, it is contemplated that the present disclosure is not necessarily limited to these aspects. In particular, it should be understood that while specific embodiments of the temperature oscillation overlay device have been described with reference to particular hardware configurations, wireless protocols, temperature sensor types, and biological applications, the present disclosure is not limited to these specific implementations, and that equivalent structures, methods, and applications that achieve the same functional result of superimposing a programmable biologically defined time-varying temperature profile onto an existing tissue culture incubator without permanent modification thereof are contemplated as falling within the scope of the present disclosure.

What is claimed is:

1. A temperature oscillation overlay device configured for removable integration with an existing tissue culture incubator having a native temperature controller and a native heating element, the device comprising:
a. a temperature sensing system physically independent of the native temperature controller, the temperature sensing system comprising at least one temperature sensor configured for placement within an interior chamber of the incubator and a temperature sensing system microcontroller configured to receive data from the temperature sensor;
b. a central microcontroller configured to receive temperature measurements obtained by the at least one temperature sensor from the temperature sensing system microcontroller;
c. an independent internal heating element configured for placement within the interior chamber of the incubator and operated entirely separately from and without modification of the native heating element or native temperature controller;
d. a power modulation interface comprising a power modulation interface microcontroller configured to selectively modulate electrical power delivered exclusively to the independent internal heating element without modification of the incubator's internal control circuitry; and
e. a programmable waveform engine executed by the central microcontroller and configured to generate a biologically defined time-varying temperature profile;
wherein the central microcontroller controls power delivery to the independent internal heating element via the power modulation interface in a closed feedback loop independent of the native temperature controller to superimpose the oscillatory temperature profile wherein temperature increases are achieved by increasing power delivery to the independent internal heating element and temperature decreases are achieved by reducing or ceasing power delivery to the independent internal heating element, with the rate of passive thermal equilibration toward the stable thermal baseline controlled by the central microcontroller to conform to the descending phases of the biologically defined time-varying temperature profile.

2. The device of claim 1, wherein the biologically defined time-varying temperature profile is representative of a circadian rhythm, an ultradian rhythm, an infradian rhythm, a fever profile, or a hypothermic profile.

3. The device of claim 1, wherein the programmable waveform engine permits user-defined adjustment of waveform shape, amplitude, period, phase, and mean temperature of the biologically defined time-varying temperature profile.

4. The device of claim 1, wherein the biologically defined time-varying temperature profile comprises a circadian rhythm with a period of 18 to 30 hours and an amplitude between 0.5° C. and 3° C.

5. The device of claim 1, wherein the device further comprises a plurality of wireless communication modules operatively coupled to the central microcontroller, the temperature sensing system microcontroller, and the power modulation interface microcontroller to effectuate wireless communication between the temperature sensing system microcontroller and the central microcontroller and between the central microcontroller and the power modulation interface microcontroller.

6. The device of claim 5, wherein the wireless communication modules operate using a peer-to-peer protocol configured for direct device-to-device communication without reliance on external network infrastructure.

7. The device of claim 6, wherein the peer-to-peer protocol comprises ESP-NOW protocol providing greater than 99% connectivity reliability.

8. The device of claim 1, wherein the device further comprises one or more data storage modules communicatively coupled to the temperature sensing system microcontroller, wherein the data storage module is configured to store timestamped temperature data independent of wireless connectivity.

9. The device of claim 1, wherein the at least one temperature sensor comprises a platinum resistance temperature detector providing temperature measurements with a precision of approximately ±0.2° C. across an operating range of 30° C. to 50° C.

10. The device of claim 1, wherein the central microcontroller maintains temperature within the tissue culture incubator within ±0.3° C. of a programmed setpoint as determined by the time-varying temperature profile.

11. The device of claim 1, wherein the programmable waveform engine is communicatively coupled to a graphical user interface permitting programming of the time-varying temperature profile and real-time display of temperature parameters.

12. The device of claim 1, wherein the central controller is configured to receive temperature measurements from a plurality of tissue culture incubators and relay instructions to a plurality of power modulation interfaces associated with the plurality of tissue culture incubators to modulate power delivery to a plurality of independent internal heating elements.

13. The device of claim 1, wherein the central microprocessor provides real-time remote monitoring and alert notification upon deviation from programmed temperature parameters.

14. The device of claim 1, wherein the device further comprises an auxiliary temperature control unit under control of the central microcontroller to augment heating from the independent internal heating element, wherein temperature reduction is achieved passively by reducing or ceasing power delivery to the independent internal heating element, with the rate of passive thermal equilibration toward the stable thermal baseline actively controlled by the central microcontroller via the closed feedback loop to conform to the descending phases of the biologically defined time-varying temperature profile.

15. A method of entraining circadian gene expression in cultured cells, comprising:
a. maintaining the cells in a standard tissue culture incubator;
b. superimposing using the temperature oscillation overlay device of claim 1 a continuous oscillatory temperature profile having a period of 20 to 28 hours and an amplitude between 0.5° C. and 3° C.; and
c. maintaining the oscillatory temperature profile for at least five consecutive circadian cycles,
wherein the oscillatory temperature profile functions as a sustained zeitgeber that increases circadian amplitude and reduces inter-replicate variability relative to serum shock synchronization.

16. The method of claim 15, wherein the cultured cells are mammalian.

17. The method of claim 15, wherein the oscillatory temperature profile has an amplitude of ±1 to 2° C. relative to a mean temperature of 37° C.

18. the method of claim 15, wherein the oscillatory temperature profile is maintained for at least ten consecutive circadian cycles.

19. A method of detecting circadian regulatory elements in a gene promoter, comprising:
a. introducing into cultured cells a reporter construct comprising a promoter region operatively linked to a reporter gene;
b. maintaining the cultured cells in a standard tissue culture incubator with the native temperature controller set to the minimum temperature of the desired oscillation profile to establish a stable thermal baseline;
c. superimposing using the temperature oscillation overlay device of any of claims 1 through 14 a continuous oscillatory temperature profile having a period of 18 to 30 and an amplitude between 0.5° C. and 3° C.;
d. maintaining the oscillatory temperature profile for at least five consecutive circadian cycles; and
e. measuring reporter gene expression over time,
wherein differences in amplitude, period, acrophase, or baseline expression of reporter gene activity under the superimposed continuous oscillatory temperature profile identify the presence or functional contribution of circadian regulatory elements.

20. The method of claim 19, wherein the reporter gene comprises a luciferase gene, a fluorescent protein reporter gene, or an enzymatic reporter gene.

21. The method of claim 19, wherein the circadian regulatory element comprises an E-box sequence, a D-box sequence, or an RRE sequence.
